SEQUENCE LISTING (6 sequences)

SEQ ID NO: 1 - DNA, 24 bp, Artificial Sequence
  Synthetic oligonucleotide
ggcacccagcacaatgaagatcaa

SEQ ID NO: 2 - DNA, 24 bp, Artificial Sequence
  Synthetic oligonucleotide
tagaagcatttgcggtggacgatg

SEQ ID NO: 3 - DNA, 24 bp, Artificial Sequence
  Synthetic oligonucleotide
tcacagcctgcagacacaactcaa

SEQ ID NO: 4 - DNA, 24 bp, Artificial Sequence
  Synthetic oligonucleotide
ccaaactggctttgcagatgctga

SEQ ID NO: 5 - DNA, 22 bp, Artificial Sequence
  Synthetic oligonucleotide
gatggctcagtggacttcaacc

SEQ ID NO: 6 - DNA, 22 bp, Artificial Sequence
  Synthetic oligonucleotide
tgctatgcaccttctccagacc

(19) United States
(12) Patent Application Publication
(10) Pub. No.: US 2018/0203010 A1
(43) Pub. Date: Jul. 19, 2018

(54) ANGPTL4 AS A PROGNOSTIC MARKER FOR BLADDER CANCER

(71) Applicants: Rita Ghosh, San Antonio, TX (US); Tiffani Houston, San Antonio, TX (US); Neelam Mukherjee, San Antonio, TX (US); Roble G. Bedolla, San Antonio, TX (US); Eduardo Cardenas, San Antonio, TX (US)

(72) Inventors: Rita Ghosh, San Antonio, TX (US); Tiffani Houston, San Antonio, TX (US); Neelam Mukherjee, San Antonio, TX (US); Roble G. Bedolla, San Antonio, TX (US); Eduardo Cardenas, San Antonio, TX (US)

(73) Assignee: THE BOARD OF REGENTS OF THE UNIVERSITY OF TEXAS SYSTEM, Austin, TX (US)

(21) Appl. No.: 15/873,593

(22) Filed: Jan. 17, 2018

Related U.S. Application Data
(60) Provisional application No. 62/447,013, filed on Jan. 17, 2017.

(51) Int. Cl. G01N 33/574

(52) U.S. Cl. CPC G01N 33/57407; G01N 2333/515

(57) ABSTRACT

Certain embodiments are directed to methods and compositions for detecting or measuring ANGPTL4 in urinary bladder cancer sample from a subject and assessing the bladder cancer status of the subject.

### PRIORITY CLAIM

This application claims priority to U.S. Application No. 62/447,013 filed Jan. 17, 2017, which is incorporated by reference in its entirety.

### STATEMENT REGARDING FEDERALLY FUNDED RESEARCH

This invention was made with government support under CA129516 and CA149516 awarded by the National Cancer Institute. The government has certain rights in the invention.

### REFERENCE TO SEQUENCE LISTING

A sequence listing required by 37 CFR 1.821-1.825 is being submitted electronically with this application. The sequence listing is incorporated herein by reference.

### BACKGROUND

Bladder cancer is the second most common and the second leading cause of mortality among urologic malignancies (Enokida and Nakagawa, International Journal of Clinical Oncology 2008, 13:298-307). 70%-80% of bladder tumors are non-invasive, with a recurrence rate of up to 70% with 10%-15% of recurrent tumors progressing to muscle invasion and metastasis (Holmang et al., J Urol 1995, 153:1823-26; Nativ et al., J Urol 2009, 182:1313-17). Long-term, vigilant surveillance and aggressive treatment plans cause bladder cancer to be one of the most expensive cancers to treat per capita, producing an estimated 3 billion dollar annual cost to the health care system (Hong et al., Urology 2008, 71:131-35). Bladder cancer is most often diagnosed in individuals over the age of 55 years old. Improved treatment and prevention is greatly needed, especially when considering the aging population of the United States. The need for a better understanding of the pathological mechanisms is pertinent in the development of more effective treatment options and preventive measures for this cancer.

Capsaicin, the bioactive polyphenol in chili peppers, inhibits growth of bladder cancer cells through various mechanisms including induction of reactive oxygen species and inhibition of cell cycle progression resulting in apoptosis induction, inhibition of proliferation and tumor cell metabolism (Chen et al., The Chinese Journal of Physiology 2012, 55:82-90; Zheng et al., Molecular Medicine Reports 2016, 13:881-87; Schwartz et al. Investigational New Drugs 2013, 31:256-64; Yang et al., Cell Metabolism 2010, 12:130-41). More recently, capsaicin-mediated apoptosis was shown to activate dendritic cells in human bladder cancer cells (Gilardini et al., Nutrition 2015, 31:578-81).

### SUMMARY

A genome-wide expression analysis was used following treatment of bladder cancer cells with capsaicin and found significant upregulation of Angiopoietin-like 4 (ANGPTL4) compared with solvent control cells. Since the literature suggests cancer protective and stimulatory roles for ANGPTL4 in various tumor models, studies were conducted to further understand whether the increased expression of ANGPTL4 in bladder cancer cells functioned to facilitate or constrain bladder cancer progression.

ANGPTL4 is a glycosylated adipokine and is a member of the angiopoietin-like (ANGPTL) family that consists of 7 ANGPTL proteins (Hato et al., Trends Cardiovasc Med 2008, 18:6-14; Katoh et al., Int J Mol Med 2006, 17: 1145-49). The native full-length ANGPTL4 (flANGPTL4) protein exists as a dimeric or tetrameric complex that is proteolytically cleaved by pro-protein convertases to give rise to an N-terminal coiled-coil fragment (nANGPTL4) and a C-terminal fibrinogen-like domain (cANGPTL4) (Lei et al., The Journal of Biological Chemistry 2011, 286:15747-56). The limited yet conflicting reports of the biological role of ANGPTL4 in cancer suggest it has both pro- and anti-tumor roles (Ma et al., PNAS U.S.A. 2010, 107:14363-68; Ito et al., Cancer Research 2003, 63:6651-57; Padua et al., Cell 2008, 133:66-77; Galaup et al., PNAS U.S.A. 2006, 103:18721-26; Le Jan et al., The American Journal of Pathology 2003, 162:1521-28). fl-ANGPTL4 has been shown to bind heparin sulfate proteoglycans resulting in the inhibition of endothelial cell migration and tubule formation (Cazes et al., Circ Res 2006, 99:1207-15). The cleaved carboxy terminus of ANGPTL4 (cANGPTL4) has been shown to bind and activate integrins β1 and β5 to modulate cell migration through regulation of focal adhesion kinase (FAK)/p21-activated kinase (PAK)-signaling pathway (Goh et al., Am J Pathol 2010, 177:2791-803). cANGPTL4 also binds to vascular-endothelial cadherin (VE-Cad) and claudin-5 at endothelial junctions, resulting in vascular disruption by activating integrin α5β1 (Huang et al., Blood 2011, 118:3990-4002). On the contrary, presence of ANGPTL4 was associated with gastric and colorectal cancer venous invasion (Nakayama et al., Oncology Reports 2010, 24:599-606).

Here the association of ANGPTL4 with histological grade and stage (muscle invasiveness) of urinary bladder cancer is described. Examination of various biological endpoints associated with bladder cancer using cell lines following modulation of ANGPTL4 expression showed for the first time that ANGPTL4 is involved in bladder cancer cell motility through cytoskeletal organization of the urothelium but not its proliferative potential. Since ANGPTL4 is an adipokine the results have implications for the involvement of adipokine-mediated inflammation in bladder cancer.

Certain embodiments are directed to methods for qualifying bladder cancer status in a subject comprising measuring ANGPTL4 in a biological sample from the subject. In certain aspects nuclear, secreted, or nuclear and secreted ANGPTL4 is measured. In a further aspect full length ANGPTL4 is measured. In certain aspects a carboxy terminal segment of ANGPTL4 is measured. In certain instances the biological sample can be a bladder tissue sample. ANGPTL4 is measured can be measured or detected by immunoassay. In certain aspects ANGPTL4 is measured by immunohistochemistry. An elevated level of ANGPTL4 can indicate a favorable prognostic bladder cancer status. The method can further comprise managing subject treatment based on the status.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIGS. 1A-1F. Differential ANGPTL4 message and protein levels in bladder tumors. (A) Human bladder cancer cDNA array containing cDNA from 24 tissue samples representing different histological grades was used to determine ANGPTL4 mRNA expression in normal tissues (n=4), G1 tumors (n=3), G2-3 tumors (n=15), and G4 tumors (n=2). Immunohistochemistry in the bladder TMAs to examine ANGPTL4 protein level in normal urothelium (n=82) vs CIS (B; n=23), low-grade papillary urothelial neoplasm C; n=51), non-invasive high-grade papillary urothelial carcinoma (D; n=24)(or non-muscle-invasive high-grade urothelial carcinoma (D; n=27)), or invasive high-grade urothelial carcinoma (E; n=54)(or muscle-invasive highgrade urothelial carcinoma (E; n=51)), as well as in (F) non-muscle-invasive (n=78) vs muscle-invasive (n=51) tumors.

FIGS. 2A-2D. Various urothelial cell lines have differential ANGPTL4 message and protein expression. (A) Quantitative polymerase chain reaction analysis of ANGPTL4 mRNA. (B) Full-length and (C) c-terminus fragment of ANGPTL4 protein levels. (D) ELISA analysis of secreted full-length ANGPTL4.

FIGS. 3A-3C. ANGPTL4 does not affect bladder cancer cell viability or capacity to proliferate. (A) MTT assay measuring proliferation on day 2, 3 and 4 post-transfection of pANGPTL4 in T24 and TCCSUP bladder cancer cells. (B) Trypan blue assay measuring cell viability on days 2, 3 and 4 post-transfection. (C) Immunoblot confirming ANGPTL4 over-expression on days 2, 3, and 4 post-transfection in T24 and TCCSUP bladder cancer cell lines.

FIGS. 4A-4D. ANGPTL4 inhibits bladder cancer cell migration, motility and invasion in bladder cancer cell lines. Transwell invasion assay measuring cell ability to invade through the basement membrane in (A) T24 cells and (B) TCCSUP cells overexpressing ANGPTL4 (Ai and Bi) or in wild type cells assayed in conditioned media from ANPTL4 overexpressing cells maintained in serum free media (Aii and Bii); immunoblot to confirm ANGPTL4 overexpression (Aiii and Biii). Motility in (C) T24 cells and (D) TCCSUP cells overexpressing ANGPTL4 (Ci and Di) or in wild type cells assayed in conditioned media from ANGPTL4 overexpressing cells (Cii and Dii). Ability to migrate through Transwell porous insert assayed in conditioned media from ANGPTL4 overexpressing cells maintained in serum free media (Ciii and Diii). Figures represent ≥3 independent experiments, *p<0.05 (Students T-test).

FIGS. 5A-5B. ANGPTL4 alters cytoskeletal organization in bladder cancer cells. Confocal microscopy following F-actin staining with rhodamine-phalloidin in (A) T24 and (B) TCCSUP bladder cancer cell lines reveals differences in actin organization between vector transfected and pANGPTL4 transfected cells.

FIGS. 6A-6C. Cytoskeletal signaling pathway modulated by ANGPTL4. (A) Basal levels of cytoskeletal signaling proteins in T24 and TCCSUP cells. (B) Changes in cytoskeletal signaling following forced expression of ANGPTL4 in T24 cells and (C) TCCSUP cells.

FIG. 7. Transient ANGPTL4 knockdown does not affect normal urothelial cell viability or capacity to proliferate. Immunoblot confirming ANGPTL4 knockdown on days 3 and 5 post-transfection in HB1EC normal bladder cell line. Trypan blue assay measuring cell viability on days 3, 4 and 5 post-transfection.

FIGS. 8A-8B. ANGPTL4 inhibits cell migration and motility in low-grade, but not high-grade bladder cancer cell lines. Wound scratch assay images in (A) T24 (A) and (B) TCCSUP cell lines either overexpressing ANGPTL4 (left) or in wild type cells assayed in conditioned media from ANGPTL4 overexpressing cells (right).

FIGS. 9A-9B. F-actin organization in bladder cancer cell lines. Rhodamine-phalloidin staining to show F-actin organization in (A) T24 and (B) TCCSUP cell lines.

FIGS. 10A-10B. Quantification of cytoskeletal signaling pathway modulated by ANGPTL4 in T24 cells. Quantification of immunoblotting data to assess changes in activation of (A) FAK and (B) Src proteins following ectopic expression of ANGPTL4 in T24 cells. Each time point quantified as pANGPTL4/pCMV.

FIGS. 11A-11B. Quantification of cytoskeletal signaling pathway modulated by ANGPTL4 in TCCSUP cells. Quantification of immunoblotting data to assess changes in activation of (A) FAK and (B) Src proteins following ectopic expression of ANGPTL4 in TCCSUP cells. Each time point quantified as pANGPTL4/pCMV.

### DESCRIPTION

The phenolic compound capsaicin (8-methyl-N-vanillyl-6-nonenamide) induces apoptosis through various mechanisms to inhibit urinary bladder cancer cell growth. The inventors used whole genome expression analysis and found robust upregulation of Angiopoietin-like 4 (ANGPTL4) in bladder cancer cells treated with capsaicin. ANGPTL4 is a serum hormone glycoprotein and its role in bladder cancer has not been investigated. The clinical significance of the observation was established using immunohistochemistry to stain bladder tissue microarrays for ANGPTL4. Differential ANGPTL4 levels were identified between non-neoplastic urothelial tissues and urothelial tumors and carcinoma in situ. To understand the biological role of ANGPTL4 in bladder cancer bladder cancer cells derived from aggressive non-metastatic and metastatic tumors were used. Forced expression of ANGPTL4 in bladder cancer cells decreased cell motility through the FAK/SRC pathway that culminated in the reorganization of the actin cytoskeleton. Since ANGPTL4 regulates glucose homeostasis, insulin sensitivity, and lipid metabolism, it was proposed that ANGPTL4 may serve to bridge metabolic syndrome-related molecular changes with progression of urothelial cancer. Understanding the role of ANGPTL4 in bladder cancer development and progression would facilitate the exploitation of ANGPTL4 for clinical use. Log rank test of Kaplan-Meier analysis showed a trend towards cancer-free survival, recurrence-free survival of non-muscle invasive tumors, and progression-free survival of muscle invasive tumors in the presence of ANGPTL4 than without ANGPTL4 expression. Since ANGPTL4 is a secreted protein, the differential level of ANGPTL4 can potentially serve as a prognostic marker for urothelial cancer progression.

In seeking novel therapeutic options for bladder cancer the adipokine ANGPTL4 was identified as a protein that is induced upon capsaicin treatment. Previous studies on ANGPTL4 have largely focused on its role in the regulation of lipid homeostasis through direct inhibition of lipoprotein lipase. Currently there is still controversy regarding the role of ANGPTL4 in cancer and data is lacking regarding its role in bladder cancer (Ito et al., Cancer Research 2003, 63:6651-57; Galaup et al., PNAS U.S.A. 2006, 103:18721-26; Le Jan et al., The American Journal of Pathology 2003, 162:1521-28; Cazes et al., Circ Res 2006, 99:1207-15; Goh et al., Am J Pathol 2010, 177:2791-803; Huang et al., Blood 2011, 118:3990-4002; Zhu et al., Cancer Cell 2011, 19:401-15; Manuvakhova et al., Journal of Neuroscience Research 2011, 89:58-72; Goh et al., The Journal of Biological Chemistry 2010, 285:32999-3009). The relevant role of ANGPTL4 in bladder cancer is described herein.

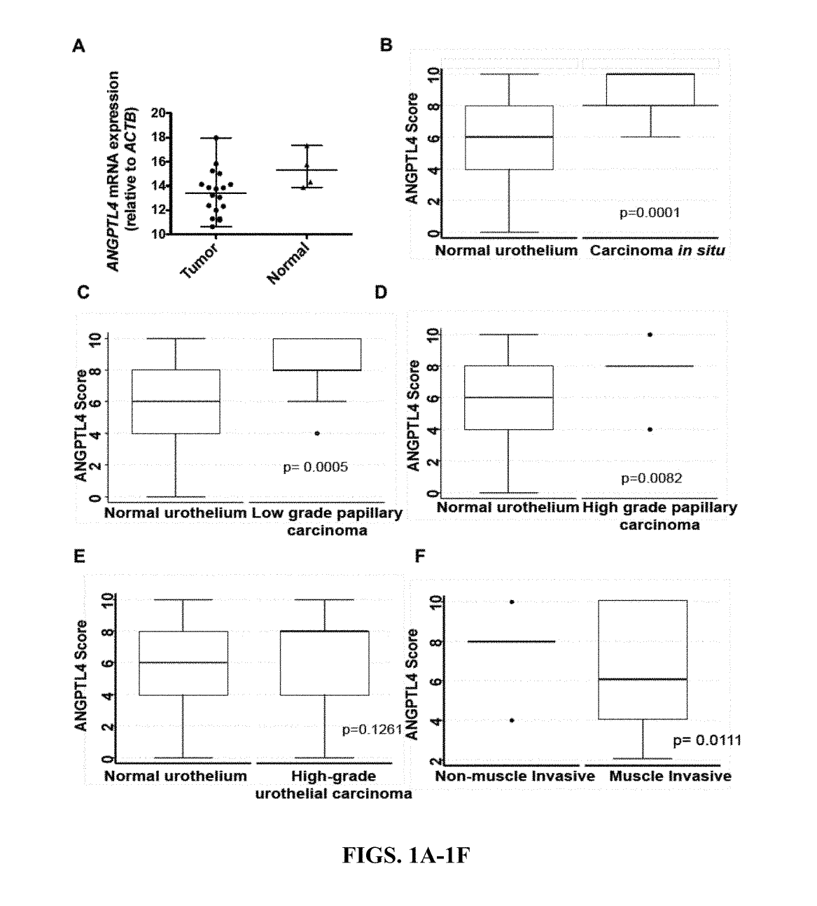

The human cDNA array and TMA data showed a pattern indicative of a complex model of ANGPTL4 expression and its role in bladder cancer and tumor progression. ANGPTL4 expression decreased during bladder cancer transition from low grade into high grade tumor (FIG. 1). ANGPTL4 may function early in cancer development, morphology of non-invasive versus muscle-invasive high-grade carcinomas is similar but different from that of normal tissue. This also marks a time of increased proliferation. As tumors progress and outgrow nutritional resources and become increasingly hypoxic, angiogenesis and invasion are mechanisms a tumor uses to evade growth restrictions and survive. ANGPTL4 may inhibit this process and thus loss of expression may promote loss of differentiation, angiogenesis, and cells ability to invade to nearby and distant organs. ANGPTL4 has previously been shown to modulate these processes (Ito et al., Cancer Research 2003, 63:6651-57; Galaup et al., PNAS U.S.A. 2006, 103:18721-26; Le Jan et al., The American Journal of Pathology 2003, 162:1521-28; Cazes et al., Circ Res 2006, 99:1207-15; Goh et al., Am J Pathol 2010, 177:2791-803; Huang et al., Blood 2011, 118:3990-4002; Zhu et al., Cancer Cell 2011, 19:401-15; Manuvakhova et al., Journal of Neuroscience Research 2011, 89:58-72; Goh et al., The Journal of Biological Chemistry 2010, 285:32999-3009). These results suggest a unique context/temporal specific function of ANGPTL4. The current view is that bladder cancer arises from two pathways distinguishing between low-grade papillary carcinomas and CIS. Both are early stage, however, CIS has a poorer prognosis and has a higher likelihood of progression into invasive and metastatic disease (Ewald et al., PloS one 2013, 8:e55414; McConkey et al., Urol Oncol 2010, 28:429-40; Morrison et al., PNAS U.S.A. 2014, 111:E672-81; Wu et al., Nat Rev Cancer 2005, 5:713-25).

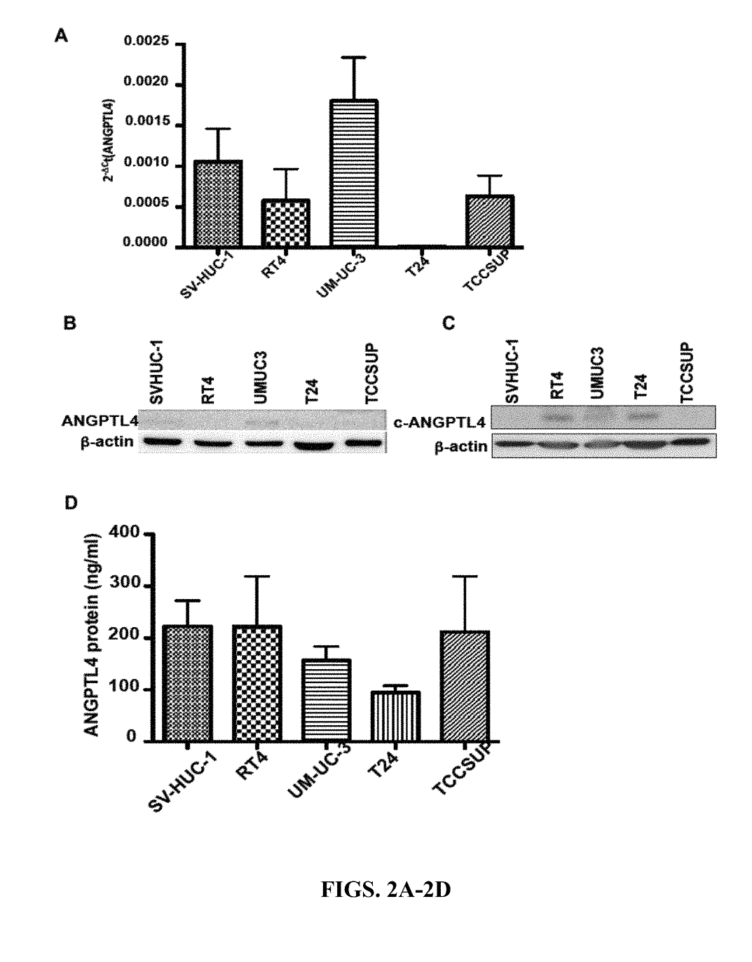

ANGPTL4 has been shown to be silenced in mammary carcinomas, gastric cancers and has been shown to have tumor suppressive characteristics in numerous studies (Ito et al., Cancer Research 2003, 63:6651-57; Galaup et al., PNAS U.S.A. 2006, 103:18721-26; Cazes et al., Circ Res 2006, 99:1207-15; Arao et al., Int J Cancer 2006, 118:483-89; Hattori et al., Cancer Sci 2011, 102:1337-43; Kaneda et al., Cancer Res 2002, 62:6645-50). ANGPTL4 is both secreted and cleaved by proteases rendering an amino terminal nANGPTL4 and carvboxy terminal cANGPTL4 (Lei et al., The Journal of Biological Chemistry 2011, 286:15747-56). Both the full length flANGPTL4 and nANGPTL4 are known to function in lipid metabolism (Sukonina et al., PNAS U.S.A. 2006, 103:17450-55; Mandard et al., The Journal of Biological Chemistry 2006, 281:934-44; Georgiadi et al., Circ Res 2010, 106:1712-21). Some reports indicate that the cANGPTL4 possesses tumor suppressive capacity due to its ability to inhibit the RAF/ERK pathway stimulation of angiogenesis. On the other hand, it has been shown that cANGPTL4 contributes to a pro-survival phenotype in which ANGPTL4 increased O₂:H₂O₂ ratio and anoikis resistance (Yang et al., Arterioscler Thromb Vasc Biol 2008, 28:835-40; Zhu et al., Cancer Cell 2011, 19:401-15). T24 and TCCSUP cell lines were chosen for further study as they represent moderately differentiated (non-metastatic) and undifferentiated (metastatic) tumors, respectively. Basal levels of cANGPTL4 in the human bladder cell line panel did not reveal any correlation that pointed towards an obvious role of cANGPTL4 in bladder cancer cells (FIG. 2C). Elevated levels of cANGPTL4 were detected in T24 relative to TCCSUP (FIGS. 2A and 2D). It was contemplated that a fundamental alteration in ANGPTL4 processing that leads to an increase in cANGPTL4 protein levels in T24 even though mRNA and ELISA data indicate TCCSUP cells have higher flANGPTL4 level.

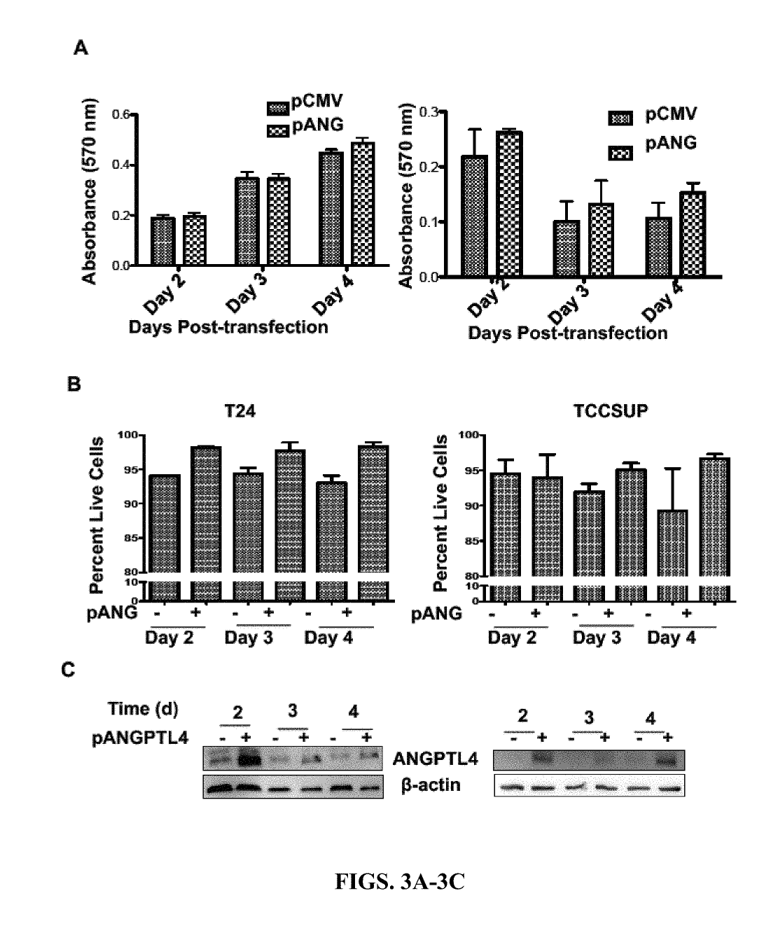

Overexpressing ANGPTL4 in T24 and TCCSUP did not result in significant changes on cell viability or proliferation as previous research has shown (FIG. 3) (Ito et al., Cancer Research 2003, 63:6651-57; Kim et al., Cancer Res 2011, 71:7010-20). It is shown that ANGPTL4 affects the ability to migrate and invade as reported by numerous studies in hepatocellular and breast carcinomas, melanoma/keratinocytes, human umbilical vein endothelial cells and colon cancer cell lines (Padua et al., Cell 2008, 133:66-77; Galaup et al., PNAS U.S.A. 2006, 103:18721-26; Cazes et al., Circ Res 2006, 99:1207-15; Goh et al., Am J Pathol 2010, 177:2791-803; Nakayama et al., Oncology Reports 2010, 24:599-606; Huang et al., Oncol Rep 2012, 27:1541-47; Manuvakhova et al., Journal of Neuroscience Research 2011, 89:58-72; Zhang et al., Oncogene 2012, 31:1757-70; Goh et al., The Journal of Biological Chemistry 2010, 285:32999-3009).

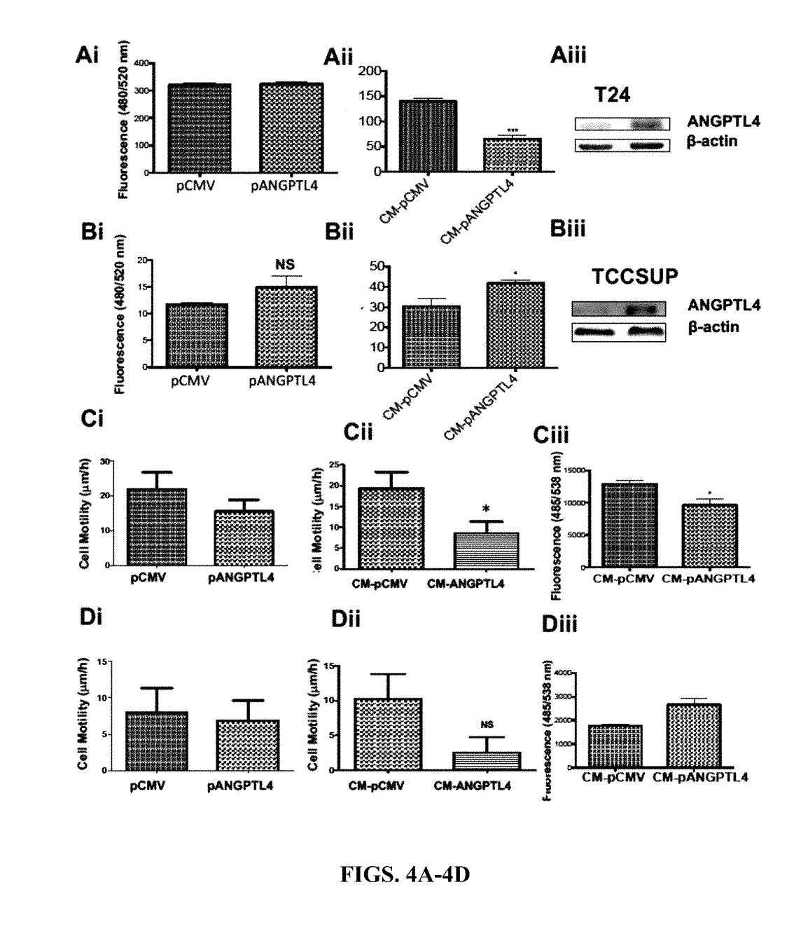

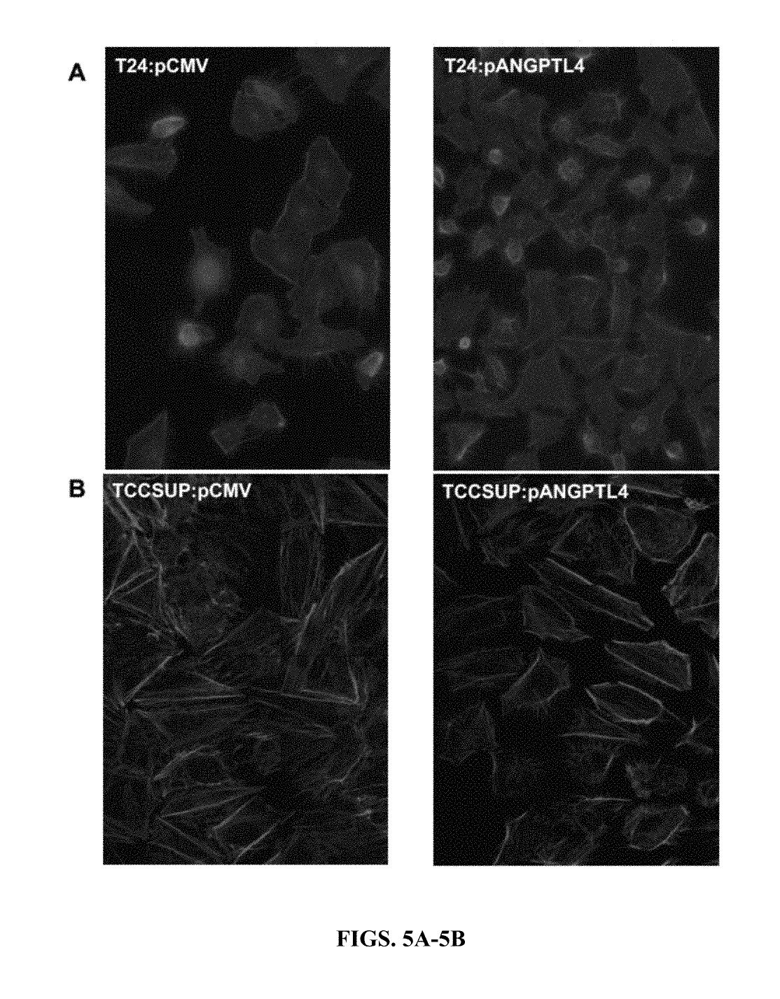

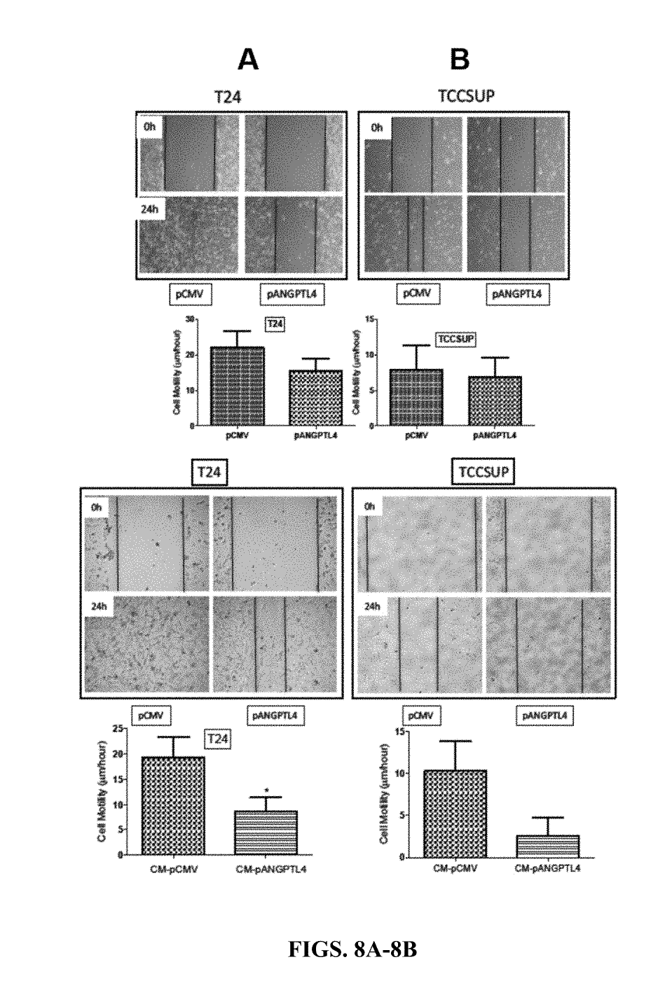

In overexpressing ANGPTL4 in T24 and TCCSUP cells no effect was seen on invasion (FIGS. 4A and 4B). It is contemplated that there is a differential processing or response to ANGPTL4 expression between the two cell lines. Recognizing that ANGPTL4 can function as a secreted protein, a trans-well invasion assay was performed on non-transfected cells grown in the conditioned media of transfected T24 and TCCSUP cells. This resulted in a significant inhibition of the ability of T24 cells to invade through the basement membrane (FIG. 4A). The same held true in wound scratch and transwell migration assays in which only T24 cells ability to close the gap was significantly impaired in response to conditioned media from T24 cells ectopically overexpressing ANGPTL4 (FIG. 4C and FIG. 8C). Surprisingly an increase in invasion in TCCSUP cells was observed and a small but insignificant inhibition in cell motility and migration when using the conditioned media in TCCSUP (FIG. 4D and FIG. 8B). This implies that the anti-tumor effect of secreted ANGPTL4 might have a reduced role in this cell line.

The results from experiments examining F-actin organization, migration/invasion, and cytoskeleton signaling pathways lead to the conclusion that ANGPTL4 inhibits the moderately differentiated bladder cancer cell line, T24, from migrating through alteration of the actin cytoskeleton. An increased filopodia formation was seen in T24 ANGPTL4 overexpressing cells and, to some extent, increased lamellipodia and stress fiber formation (FIG. 5). These are characteristics typically associated with motile cells. Yet in order for a cell to have dynamic and consistent movement, constant turnover of actin polymerization and depolymerization must be achieved.

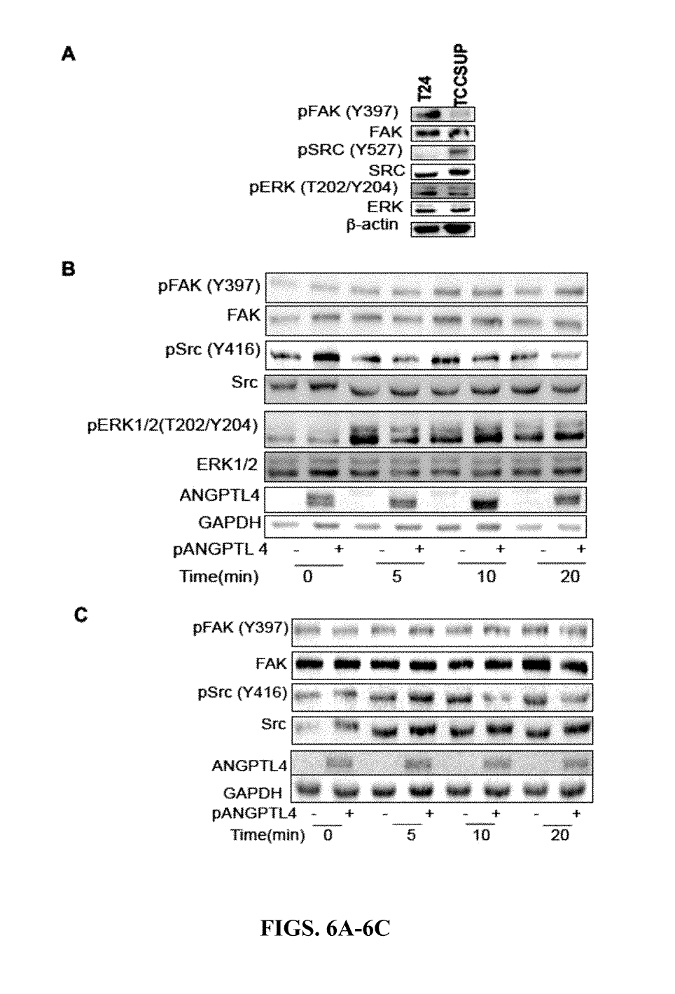

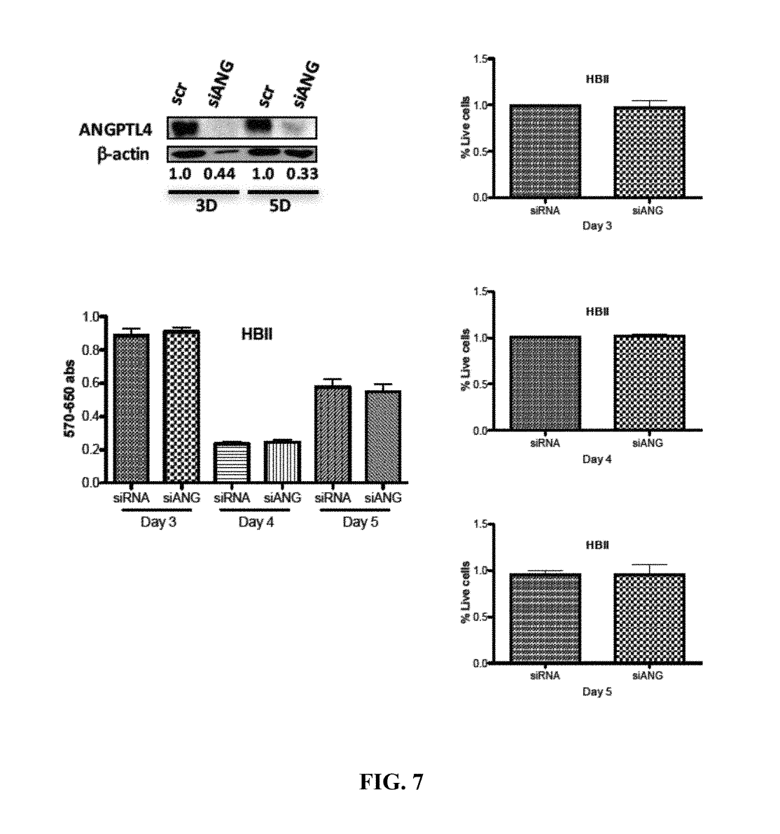

The data indicate this key turnover mechanism is likely lacking in T24 cells. Overexpressing ANGPTL4 increased phosphorylation of upstream cytoskeletal signaling protein FAK in T24 cells. FAK functions in multiple signaling pathways to alter migration and cytoskeletal reorganization through both its kinase activity and via its adaptor protein function while Src, on the other hand, is crucial to cell adhesion turnover and thus motility and migration (Carragher et al., Trends Cell Biol 2004, 14:241-49; Cary et al., Mol Cell Biol 2002, 22:2427-40; Fincham et al., EMBO J 1998, 17:81-92; Li et al., Mol Cell Biol 2002, 22:1203-17; Sieg et al., Nat Cell Biol 2000, 2:249-56; Zhai et al., The Journal of Biological Chemistry 2003, 278:24865-73). The immunoblot data, in which ANGPTL4 is overexpressed in T24 cells showed increased active FAK, yet decreased activated Src (FIG. 6). These results suggest that although increased level flANGPTL4 may increase FAK signaling, activation of Src is decreased and thus results in decreased cell adhesion turnover and the inhibition of motility in the T24 cells. ANGPTL4 overexpression decreased activated FAK and Src in the TCCSUP cells and did not translate into significant motility inhibition. This provides further evidence that the role of ANGPTL4 as a barrier for metastasis is less important in poorly differentiated bladder cancer. Phosphorylated FAK is closely associated with F-actin dynamics and this may be why the change in organization of F-actin and the lamellipodia-like phenotype in these cells was seen.

### I. BIOMARKERS

A biomarker is an organic biomolecule, the presence of which in a sample is used to determine the phenotypic status of the subject, tissue, or cell (e.g., bladder cancer patient v. normal or non-bladder cancer patient). In a preferred embodiment, the biomarker is differentially present in a sample taken from a subject of one phenotypic status (e.g., having a disease) as compared with another phenotypic status (e.g., not having the disease). A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio. Biomarkers, alone or in combination, provide measures of relative risk that a subject belongs to one phenotypic status or another. Therefore, they are useful as markers for disease (diagnostics), therapeutic effectiveness of a drug (theranostics) and drug toxicity.

Certain aspects of the invention provide polypeptide-based biomarkers that are used to distinguish subjects with bladder cancer from subjects that are normal or with non-bladder cancer. In a further aspect the biomarkers are used to evaluate and classify a subject having bladder cancer. The biomarkers are preferably differentially present in subjects having bladder cancer, versus subjects who are normal or have non-bladder cancer.

The biomarker(s) of the invention can be detected in biological fluids, such as urine or blood, or in tissue samples, such as biopsies or resections. The biomarkers can be isolated from biological fluids, such as urine or serum. They can be isolated by any method known in the art, based on both their mass and their binding characteristics. For example, a sample comprising the biomolecules can be subject to in situ hybridization or chromatographic fractionation, and optionally subject to further separation, e.g., acrylamide gel electrophoresis.

It has been found that proteins frequently exist in a sample in a plurality of different forms characterized by detectably different masses. These forms can result from pre-translational modifications, post-translational modifications or both. Pre-translational modified forms include allelic variants, splice variants and RNA editing forms. Post-translationally modified forms include forms resulting from, among other things, proteolytic cleavage (e.g., fragments of a parent protein that include amino terminal or carboxy terminal fragments or products), glycosylation, phosphorylation, lipidation, oxidation, methylation, cystinylation, sulphonation and acetylation. Modified forms of any biomarker of this invention also may be used, themselves, as biomarkers. Modified forms of a biomarker can be detected by specific binding agents that can detect and distinguish the modified from the biomarker. More specifically, the proteins are captured using biospecific capture reagents, such as antibodies that recognize the biomarker and/or modified forms of it.

### II. DETECTION OF BIOMARKERS FOR BLADDER CANCER

The biomarkers of this invention can be detected by any suitable method. Detection paradigms that can be employed to this end include optical methods. Illustrative of optical methods, in addition to microscopy, both confocal and non-confocal, are detection of fluorescence, luminescence, chemiluminescence, absorbance, reflectance, transmittance, and birefringence or refractive index.

### Detection by Immunoassay.

In certain embodiments, the biomarkers of this invention can be measured by immunoassay or immunohistochemistry. Immunoassay requires biospecific capture or detection reagents, such as antibodies, to capture or bind the biomarkers. Antibodies can be produced by methods well known in the art, e.g., by immunizing animals with the biomarkers. Biomarkers can be isolated from or detected in samples based on their binding characteristics.

### III. DETERMINATION OF SUBJECT BLADDER CANCER STATUS

The biomarkers of the invention can be used in diagnostic tests to assess bladder cancer status in a subject, e.g., to diagnose bladder cancer. The phrase “bladder cancer status” includes distinguishing, inter alia, bladder cancer v. non-bladder cancer and, in particular, bladder cancer v. non-bladder cancer normal or bladder cancer v. non-bladder cancer. Based on this status, further procedures may be indicated, including additional diagnostic tests or therapeutic procedures or regimens.

The power of a diagnostic test to correctly predict status is commonly measured as the sensitivity of the assay, the specificity of the assay or the area under a receiver operated characteristic (“ROC”) curve. Sensitivity is the percentage of true positives that are predicted by a test to be positive, while specificity is the percentage of true negatives that are predicted by a test to be negative. An ROC curve provides the sensitivity of a test as a function of 1-specificity. The greater the area under the ROC curve, the more powerful the predictive value of the test. Other useful measures of the utility of a test are positive predictive value and negative predictive value. Positive predictive value is the percentage of actual positives that test as positive. Negative predictive value is the percentage of actual negatives that test as negative.

A biomarker is individually useful in aiding in the determination of bladder cancer status. The method involves, first, measuring the selected biomarker in a subject sample using the methods described herein, and, second, comparing the measurement with a diagnostic amount or cut-off that distinguishes a positive bladder cancer status from a negative bladder cancer status. The diagnostic or prognostic amount represents a measured amount of a biomarker above which or below a subject is classified as having a particular bladder cancer status or prognosis. For example, if the biomarker is up-regulated compared to normal during bladder cancer, then a measured amount above the prognostic cutoff provides an indication of favorable prognosis for the bladder cancer. Alternatively, if the biomarker is down-regulated during bladder cancer, then a measured amount below the prognostic cutoff provides an unfavorable prognosis for the bladder cancer. As is well understood in the art, by adjusting the particular diagnostic or prognostic cut-off used in an assay, one can increase sensitivity or specificity of the assay depending on the preference of the medical professional. The particular diagnostic or prognostic cut-off can be determined, for example, by measuring the amount of the biomarker in a statistically significant number of samples from subjects with the different bladder cancer statuses and drawing the cut-off to suit the desired levels of specificity and sensitivity.

While individual biomarkers are useful biomarkers, it has been found that a combination of biomarkers can provide, greater predictive value of a particular status than single biomarkers alone. Specifically, the detection of a plurality of biomarkers in a sample can increase the sensitivity and/or specificity of the test.

In certain embodiments of the methods of qualifying bladder cancer status, the methods further comprise managing subject treatment based on the status. Such management includes the actions of the physician or clinician subsequent to determining bladder cancer status. For example, if a physician makes a diagnosis or prognosis for a bladder cancer, then a certain regime of treatment, such as prescription or administration of chemotherapy or immunotherapy might follow. Alternatively, a diagnosis or prognosis of non-bladder cancer or non-bladder cancer might be followed with further testing to determine a specific disease that might the patient might be suffering from. Also, if the diagnostic or prognostic test gives an inconclusive result on bladder cancer status, further tests may be called for.

### IV. KITS FOR DETECTION OF BIOMARKERS FOR BLADDER CANCER

In another aspect, the present invention provides kits for qualifying bladder cancer status, which kits are used to detect biomarkers according to the invention. In one embodiment, the kit comprises a capture reagent, wherein the capture reagent binds a biomarker of the invention. Thus, for example, the kits of the present invention can comprise a container comprising the biospecific capture reagent. The kit can also comprise a washing solution or instructions for making a washing solution, in which the combination of the capture reagent and the washing solution allows capture of the biomarker or biomarkers on the sample for subsequent detection. In a further embodiment, such a kit can comprise instructions for suitable operational parameters in the form of a label or separate insert. For example, the instructions may inform a consumer about how to collect the sample, how to wash the probe or the particular biomarkers to be detected. In yet another embodiment, the kit can comprise one or more containers with biomarker samples, to be used as standard(s) for calibration.

### V. USE OF BIOMARKERS FOR BLADDER CANCER IN SCREENING ASSAYS

The methods of the present invention have other applications as well. For example, the biomarkers can be used to screen for compounds that modulate the expression of the biomarkers in vitro or in vivo, which compounds in turn may be useful in treating or preventing bladder cancer in patients. In another example, the biomarkers can be used to monitor the response to treatments for bladder cancer.

The invention will be described in greater detail by way of specific examples. The following examples are offered for illustrative purposes, and are not intended to limit the invention in any manner. Those of skill in the art will readily recognize a variety of non-critical parameters that can be changed or modified to yield essentially the same results.

### VI. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

A. Materials and Methods

Cell Culture.

RT4, T24, SV-HUC-1, UM-UC-3 and TCCSUP cells were obtained from American Type Culture Collection (ATCC). RT4, T24 and TCCSUP were cultivated in the recommended ATCC culture medium. SV-HUC-1 cells were cultivated in Ham's F12K (ATCC, Manassas, Va.) media supplemented with 7% fetal bovine serum (Atlanta Biologicals, Lawrenceville, Ga.) and 1% penicillin-streptomycin (Corning Cellgro®, Manassas, Va.). UM-UC-3 cells were cultivated in minimal essential media (Corning Cellgro®) supplemented with 1 mM sodium pyruvate, 0.75% w/v sodium bicarbonate, 0.1% of 100× non-essential amino acids and 10% fetal bovine serum. The normal human bladder epithelial cell lines, HBIECI was purchased from Lifeline Cell Technology (Oceanside, Calif.) and cultured in ProstaLife™ basal media supplemented with the ProstaLife™ LifeFactors kit from LifeLine Cell Technology.

Human Bladder Tissue Microarray (TMA).

Bladder TMAs, consisting of 129 cases of urothelial neoplasm, were constructed from formalin fixed paraffin embedded specimens obtained by transurethral resection or cystectomy performed at the Johns Hopkins Hospital, as described previously (Ishiguro et al. Am J Clin Pathol 2014, 142:157-64). Appropriate approval from the institutional review board was obtained before construction and use of the TMAs. These patients included 98 men and 31 women with a mean/median age of 65.7/69 years (range: 26-89). The primary tumors included 11 papillary urothelial neoplasms of low malignant potential (PUNLMPs), 40 non-invasive (pTa) low-grade urothelial carcinomas, 27 non-muscle-invasive (pTa or pT1) high-grade urothelial carcinomas, and 51 muscle-invasive (≥pT2) high-grade urothelial carcinomas, as well as 23 concurrent urothelial carcinoma in situ (CIS) lesions from patients with muscle-invasive tumor. None of the patients had received therapy with radiation or anti-cancer drugs prior to the collection of the tissues included in the TMAs.

Immunohistochemistry.

Sections from paraffin embedded tissues were heated at 60° C., cleared in xylene and rehydrated in graded alcohols. Antigen retrieval was performed with 0.01 M citrate buffer at pH 6.0 in a 121° C. pressure chamber. Endogenous peroxidase was quenched with a TBS buffer containing 3% hydrogen peroxide followed by a protein blocking buffer incubation for 20 minutes. Each step was carried out at room temperature (22° C.). The sections were incubated for one hour at room temperature with the following antibodies: ANGPTL4 polyclonal Rabbit (Abgent Inc. San Diego Calif.). The negative control sections were incubated with a Universal Rabbit negative control Rabbit Ig fraction (DAKO Corp. Carpinteria Calif.). The ancillary and visualization systems were: Rabbit HRP polymer (BioCare Medical, Concord Calif.) and DAB Chromogen System (DAKO Corp). A total score from a range of 0 to 12 was generated based on staining grade by a single pathologist that took into account proportion and intensity of staining.

Western Blotting.

Whole cell extracts were prepared using 1× Laemmli sample buffer (4% SDS, 120 mM Tris-HCl pH 6.8, 200 μL of 0.1% bromophenol blue, 200 mM DTT to a final volume of 10 mL) with Complete™ Protease inhibitor cocktail and phosphatase inhibitor cocktail A (Santa Cruz Biotechnology, Santa Cruz, Calif.) added prior to use. Extracts were loaded on 10% SDS-PAGE and transferred electrophoretically to nitrocellulose membranes. Blocking buffer comprised of 5% non-fat dry milk in TBST (TBST-milk) was added for 1 h or overnight at 4° C. followed by 4° C. overnight incubation of primary antibody (except ANGPTL4, which was incubated at room temperature for 4 h). The following antibodies were used: ANGPTL4 (R&D Systems, Minneapolis), phosphop42/p44, phospho-AKT (Thr308/s473), p-ERK1/2(Thr202/Tyr204), ERK1/2, FAK and p-FAK, SRC and p-SRC (Cell Signaling Technology, Danvers, Mass.), ERK1 and GAPDH (Santa Cruz Biotechnology), β-actin (Sigma, St. Louis, Mo.). C-ANGPTL4 rabbit monoclonal primary antibody was a gift from Dr. Nguan Soon Tan (Nanyang Technological University, Singapore).

Transient Transfection.

TCCSUP cells were transfected with ANGPTL4-DDK or pCMV6-empty vector (Origene, Rockville, Md.). HBIEC siRNA mediated knockdown of ANGPTL4 was performed with Trilence-27 siRNA knockdown duplexes (Origene). Knockdown and over-expression was done following the manufacturers recommendations. Proteins were extracted 3 days post-transfection after 18 h serum starvation for all cells except T24; T24 signaling after short time course was performed at day 2 post-transfection. For whole cell protein extraction, 1×10⁵ cells were plated in 6-well plates. Whole cell extracts were fractionated by SDS-PAGE electrophoresis. Overexpression was verified with anti-DDK and full-length ANGPTL4 antibodies.

RNA Extraction and Quantitative Real Time PCR.

Total RNA was extracted using TRIzol® (Life Technologies-ThermoFisher, Waltham, Mass.). RNA measurements and quality were analyzed with the NanoDrop spectrophotometer (ThermoFisher Scientific, Waltham, Mass.). SuperScript®VILO™ cDNA synthesis kit (Life Technologies-ThermoFisher, Waltham, Mass.) was used to transcribe 2 μg of total RNA. The resulting cDNA was diluted 1:3 for subsequent quantitative PCR reactions using SYBR®Green PCR master mix (Applied Biosystems-ThermoFisher, Waltham, Mass.). qPCR reaction was executed in the ABI PRISM 7300 Real Time PCR system and Ct values were obtained from ABI PRISM 7300 Sequence Detector software by negative correlation with ROX dye serving as an internal reference control. Normalization was performed using β-ACTIN as a reference. Values are expressed in terms of 2Cₜ (ΔCₜ=ΔCₜₛₐₘₚₗₑ−ΔC_{tcalibrator}) or 2Cₜ (ΔCₜ=Cₜₛₐₘₚₗₑ−C_{treference}). The following primers were used at 500 nM for quantitative PCR: ACTB: forward-5′-ggcacccagcacaatgaagatcaa-3′ (SEQ ID NO:1), reverse-5′-tagaagcatttgcggtggacgatg-3′ (SEQ ID NO:2), ANGPTL4: forward-5′-tcacagcctgcagacacaactcaa-3′ (SEQ ID NO:3), reverse-5′-ccaaactggctttgcagatgctga-3′ (SEQ ID NO:4); cDNA array: TissueScan Bladder Cancer Tissue qPCR Panel I-BLRT301 cDNA array was purchased from Origene Technologies Inc. (Rockville, Md.). qPCR primers: forward-5′-gatggctcagtggacttcaacc-3′(SEQ ID NO:5), reverse-5′-tgctatgcaccttctccagacc-3′(SEQ ID NO:6).

Enzyme-Linked Immunosorbent Assay.

1×10⁵ cells/well was seeded in 6-well plates, conditioned media was harvested 24 h later and kept at −20° C. until use. Human ANGPTL4 ELISA kit was purchased from (Ray Biotech, Norcross, Ga.). ELISA was performed as per manufacturer's instructions.

Cell Viability and Proliferation Assay.

0.5-1×10⁴ cells/well was seeded in 24-well plates and transfected the next day. On day 2, 3 and 4 post-transfection cells were collected and viability was determined by trypan blue dye exclusion. 0.4-0.5×10⁵ cells/well was seeded in 96-well plate (and transfected the next day). MTT dye was added on day 2, 3, and 4 post-transfection and reduction of 4,5-dimethylthiazol-2-yl-2,5-diphenyltetetrazolium bromide (MTT) was used as an indicator of cell proliferation as described previously (Hambright et al., Molecular Carcinogenesis 2015, 54:1227-34; Hambright et al., Oncotarget 2015, 6:7195-208).

Cell Invasion.

CytoSelect™ 24-Well Invasion Assay (Cell Biolabs, San Diego, Calif.) was used according to the manufacturer's instructions. Briefly, transiently transfected cells were seeded in the upper chamber and allowed to invade the basement membrane for 24 h. Invaded cells were removed and stained with CyQuant® GR dye solution and transferred to a 96-well plate for fluorescence measurement at 480 nm/520 nm. Serum-free conditioned media was generated from T24 cells transiently transfected with pANGPTL4 or pCMV.

Wound-Scratch Assay.

2-3×10⁵ cells were plated per well in a 6-well plate and transfected the following day. A wound was created 24 h later. Cells were washed gently with PBS prior to adding charcoal stripped FBS supplemented media or serum-free media for TCCSUP and T24 respectively. Cells were visualized using the Zeiss PrimoVert microscope (Oberkochen, Germany) and images were captured at 0 h, 24 h, and 48 h using the Sony Nex 5N digital camera (Minato, Tokyo, Japan). To determine the effect of secreted ANGPTL4 on wound healing, 3×10⁵ TCCSUP and 0.5×10⁵ T24 cells were plated in 6-well plates and allowed to grow for 2 days. Wound scratch was carried out as previously described with the exception that media was replaced with serum free conditioned media from ANGPTL4 or empty vector overexpressing cells. Migration was recorded at 0 h, 24 h, and 48 h.

F-Actin Staining.

0.8×10⁵ T24 and TCCSUP cells were seeded in 35 mm culture dishes and transfected the following day with pANGPT4 or pCMV. Transfected cells were seeded on top of cover slips placed inside 35 mm culture dishes. Cells were washed twice with pre-warmed PBS (pH 7.4). Samples were then fixed in 3.7% methanol-free formaldehyde for 10 minutes and washed twice with PBS followed by acetone for 5 minutes. Cells were incubated with 1% BSA in PBS for 20 minutes at room temperature. Phalloidin-rhodamine was resuspended according to manufacturer's instructions (Life Technologies-ThermoFisher, Waltham, Mass.). Methanolic Phalloidin-rhodamine was added and coverslips were incubated in the dark for 20 minutes. DAPI was added for 10 minutes. Prairie View software (ver. 4.3) and a Nikon Eclipse FN-1 upright microscope with a 60×/1.4NA oil immersion objective was used to acquire confocal images on an Ultima confocal microscope (Prairie Technologies, Middleton, Wis.). The excitation wavelengths for DAPI and rhodamine were 405 and 561 nm, respectively. Emission bandpass filters (Semrock, Inc., Rochester, N.Y.) were 457/50 nm for DAPI and 607/70 nm for rhodamine.

Statistical Analysis.

Groups were compared with a T-test allowing for unequal variances with a Welch approximation, a Kruskal-Wallis rank test and a Wilcoxon rank-sum test as appropriate. The analysis was carried out in STATA 9.2 (STATA Co.). Values P>0.05 are considered significant. GraphPad Prism (GraphPad Software, La Jolla Calif.) was used for statistical analysis and quantitative representation of experimental data unless otherwise noted.

B. Results

Differential ANGPTL4 Message and Protein Levels in Bladder Tumors.

A human bladder cancer cDNA array was used containing cDNA from 24 tissue samples represented by different histological grades to determine ANGPTL4 expression. Real-time qPCR analysis showed that ANGPTL4 message levels increase in differentiated tumors compared with normal urothelial tissue (FIG. 1A). Comparison within the tumor samples with different grades showed that Grade 2-3 tumors had the highest ANGPTL4 expression and the least expression was seen in the Grade 4 tumors samples (FIG. 1A). A caveat of the latter observation was the small number of samples in the Grade 4 samples. These results prompted the examination of changes in ANGPTL4 protein levels in human bladder cancer samples. Immunohistochemistry of 129 bladder tissue samples showed significant increases in the ANGPTL4 level in CIS lesions (p=0.0001; FIG. 1B), low-grade tumors including PUNLMPs and low-grade papillary urothelial carcinomas (p=0.0005; FIG. 1C), and non-invasive (or non-muscle-invasive) high-grade papillary urothelial carcinomas (p=0.0082; FIG. 1D), compared with normal urothelium. Of these non-invasive (or non-muscle-invasive) tumors, CIS lesions had the highest ANGPTL4 expression. ANGPTL4 in invasive (or muscle-invasive) high-grade urothelial carcinoma samples was not significantly different from that in normal samples (p=0.126) (FIG. 1E). Further, decreased ANGPTL4 levels were found in muscle-invasive tumors compared with non-muscle-invasive tumors (p=0.0111) (FIG. 1F). These results suggest that ANGPTL4 may have important roles in bladder cancer development and progression. While increased levels of ANGPTL4 may be involved in the development of urothelial neoplasms, decreased ANGPTL4 may facilitate tumor invasion.

To identify a possible outcome of changes in ANGPTL4 in bladder cancer, a panel of human bladder epithelial cells ranging from transformed urothelial cells to cancer cell lines were screened for ANGPTL4 message and protein level. The results show a striking difference in ANGPTL4 message and protein levels in these cells. Interestingly, ANGPTL4 message was lowest in the grade III transitional cell carcinoma cell line, T24 (FIG. 2A). ELISA analysis of secreted full-length protein in these cells also reflected the lowest levels, although the cleaved and not the full-length protein was detectable in extracts from T24 cells (FIGS. 2C and 2D). ANGPTL4 is secreted and exists in its native full-length form as oligomers and as cleaved N- and C-terminal fragments. Proteolytic cleavage of ANGPTL4 and the resulting nANGTPL4 fragment is known to inhibit lipoprotein lipase activity thereby modulating its function in lipid metabolism to hydrolyze triglycerides and provide free fatty acids for cellular uptake (Lei et al., The Journal of Biological Chemistry 2011, 286:15747-56; Clement et al., Nat Med 2011, 17: 117-22; Ge et al., J Lipid Res 2005, 46:1484-90; Ge et al., The Journal of Biological Chemistry 2004, 279:2038-45; Sukonina et al., PNAS U.S.A. 2006, 103:17450-55). In contrast, studies of the cANGPTL4 have been disparate. The cANGPTL4 protein has been shown to inhibit basic fibroblast growth factor (bFGF) and VEGF-induced angiogenesis in human umbilical vein endothelial cells. In another study, cANGPTL4 was shown to disrupt endothelial integrity resulting in increased vascular leakiness and increased metastasis primarily through integrin mediated signaling (Goh et al., Am J Pathol 2010, 177:2791-803; Huang et al., Blood 2011, 118:3990-4002; Yang et al., Arterioscler Thromb Vasc Biol 2008, 28:835-40). Similarly, cANGPTL4 was found to be increased in a small sample size of tumor tissue across distinct cancer types. Furthermore, cANGPTL4 treatment in a mouse xenograft model significantly increased tumor volume compared to vehicle control (Zhu et al., Cancer Cell 2011, 19:401-15).

Contrary to what previous studies have shown in other tumor models, it was found that ANGPTL4 message and secreted full-length protein in the grade IV metastatic bladder cancer cell line, TCCSUP, were higher than the grade III cell line, T24 (FIGS. 2A and 2D). However, neither the full-length nor the cleaved protein was detectable in the extracts prepared from TCCSUP cells (FIGS. 2B and 2C). The bladder cancer patient IHC data together with the cell line results led us to hypothesize that ANGPTL4 protein may have a dual role in bladder cancer. While increased fl-ANGPTL4 protein in the urothelium may be involved in the development of papillary tumors, c-ANGPTL4 may act as a barrier to the development of metastatic disease since the metastatic patient samples and cell line had lower levels of ANGPTL4. Therefore the inventors examined if overexpressing full-length ANGPTL4 in T24 and TCCSUP cell lines would result in a change in oncogenic characteristics attributed to ANGPTL4 given the low full-length and differential cANGPTL4 protein expression in these cell lines.

ANGPTL4 does not Affect Bladder Cancer Cell Viability or Capacity to Proliferate.

Using a pCMV based plasmid vector to over-express full-length ANGPTL4 the effect of ANGPTL4 on cell viability and proliferation was evaluated. Transient overexpression of full-length ANGPTL4 in T24 and TCCSUP followed by cell viability and proliferation assays at days 2, 3 and 4 post-transfection did not confer cells a proliferative advantage or cause a significant change in cell viability (FIGS. 3A and 3B). Similarly, knocking down ANGPTL4 in normal bladder epithelial cells did not result in changes in viability or proliferative rate (FIG. 7).

ANGPTL4 Inhibits Cell Motility and Invasion in Bladder Cancer Cell Lines.

ANGPTL4 expression is linked with a cells ability to avoid anoikis, migrate, invade and metastasize to distant sites (Galaup et al., PNAS U.S.A. 2006, 103:18721-26; Goh et al., Am J Pathol 2010, 177:2791-803; Huang et al., Blood 2011, 118:3990-4002; Zhu et al., Cancer Cell 2011, 19:401-15; Huang et al., Oncol Rep 2012, 27:1541-47; Manuvakhova et al., Journal of Neuroscience Research 2011, 89:58-72; Zhang et al., Oncogene 2012, 31:1757-70). One mechanism is by full-length ANGPTL4 binding to the extracellular matrix (ECM) and interacting with heparin and heparin sulfate proteoglycans (HSPs). This interaction is shown to inhibit endothelial cell adhesion, migration and sprouting as well as alter the actin cytoskeleton (Cazes et al., Circ Res 2006, 99:1207-15; Chomel et al., FASEB Journal 2009, 23:940-49). Furthermore, the role of ANGPTL4 in cytoskeletal reorganization is shown to occur through ANGPTL4-integrin mediated signaling resulting in FAK-Src-PAK1 activation (Goh et al., Am J Pathol 2010, 177:2791-803; Huang et al., Blood 2011, 118:3990-4002; Goh et al., The Journal of Biological Chemistry 2010, 285:32999-3009). In light of these observations the effect of ANGPTL4 overexpression on T24 and TCCSUP cell motility and invasion was studied.

Transient overexpression of ANGPTL4 in T24 and TCCSUP cells did not significantly increase capacity to invade through the basement membrane (FIGS. 4A and 4B). However, when wildtype T24 cells were assayed in conditioned media from ANGPTL4 overexpressing cells the result was a significant inhibition of cells ability to invade the basement membrane (FIG. 4A). In stark contrast, TCCSUP cells exposed to conditioned media of ANGPTL4 overexpressing cells resulted in a slight increase in cell invasion (FIG. 4B). Similarly cell motility was inhibited upon transient overexpression of ANGPTL4 in T24 cells but not TCCSUP cell line (FIGS. 4C and 4D and FIG. 8). Cell motility and migration was significantly inhibited in T24 wild type cells when conditioned media from T24 cells transiently expressing ANGPTL4 was used in wound scratch and transwell migration assays (FIG. 4C). Although TCCSUP cells had a similar trend, quantification of the data did not indicate a significant inhibition of cell motility or migration (FIG. 4D).

ANGPTL4 Alters Cytoskeletal Organization.

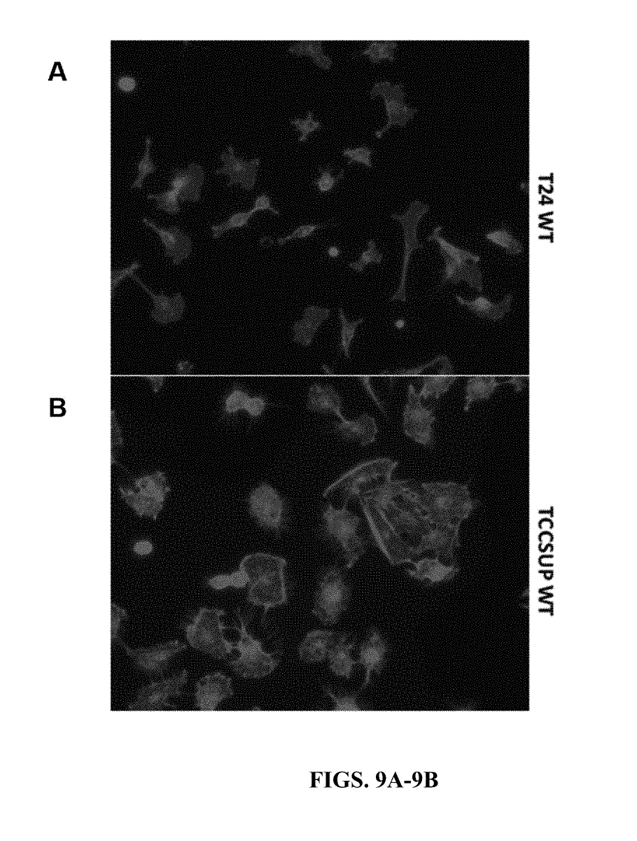

Early events in cell migration involve cytoskeletal reorganization that results morphologically as filopodia and lamellipodia formation (Le Clainche et al., Physiol Rev 2008, 88:489-513). Given the observations that overexpression of ANGPTL4 inhibits cell motility and invasion in T24 cells, ANGPTL4 role in early cytoskeletal reorganization was investigated. Firstly, upon F-actin staining with rhodamine-phalloidin it was observed that there were differences between the two non-transfected cell lines with TCCSUP cells forming defined filopodia projections and a loosely defined membrane, whereas T24 cells had a much better defined membrane and more prominent lamellipodia with little to no microspike filopodia (FIG. 9). While vector transfection did not change the organization drastically in T24 cells, transient overexpression of full-length ANGPTL4 resulted in cytoskeletal rearrangement similar in phenotype to wildtype TCCSUP with numerous filopodia projections (indicating increased cell-cell communication) and also led to a decrease in membrane definition (FIG. 5A). ANGPTL4 overexpressing TCCSUP yielded an intriguing result in that morphology resembled non-transfected T24 and TCCSUP cells with more defined cell membranes, prominent lamellipodia and filopodia (FIG. 5B).

Cytoskeletal Signaling Pathway Modulated by ANGPTL4.

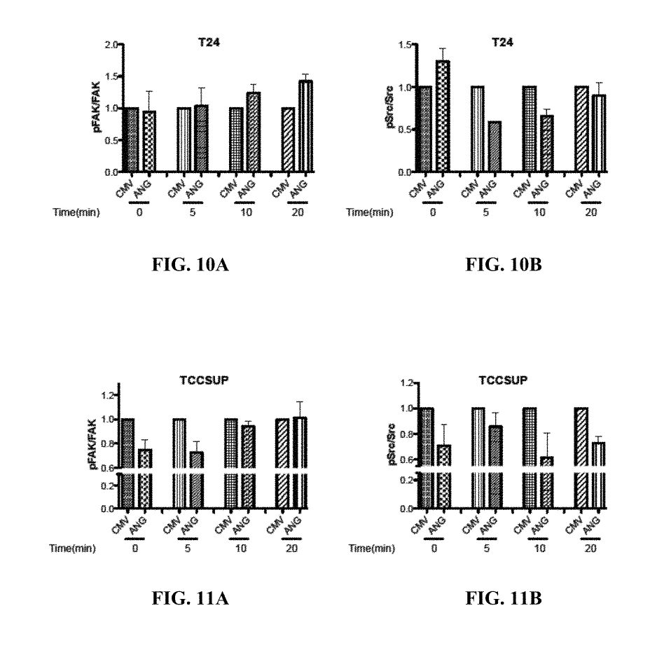

Next the signaling mechanism through which ANGPTL4 might modulate to alter cytoskeletal organization and cell motility were studied. There are several pathways that signal to the cytoskeleton including G protein coupled receptors (GPCRs), integrins, receptor tyrosine kinases (RTKs), and other specialized receptors. For example, integrins along with factors of focal adhesion (FAK) complexes couple the extracellular matrix and cytoskeleton in many cell types. Signaling through FAK-MEK pathway can terminate in lamellipodia while signaling through FAK-RAC-PAK can result in filopodia formation. Since ANGPTL4 overexpression in T24 and TCCSUP cells showed changes in lamellipodia and filopodia formation, the inventors examined the basal levels of the FAK and Src proteins in these cells. immunoblotting results show higher levels of phospho-FAK (Y397) in T24 cells compared to TCCSUP cells. Phospho-Src (Y527) was higher in TCCSUP cells and was not detectable in T24 cells (FIG. 6A). Forced-expression of ANGPTL4 led to differential changes in FAK and Src in these two cell lines. ANGPTL4 overexpression in T24 cells decreased phospho-Src (Y416) levels and increased phospho-FAK levels following addition of complete media after 18 h serum starvation (FIG. 6B and FIG. 10). Overexpression of ANGPTL4 in the TCCSUP cell line decreased the activation of both Src and FAK (FIG. 6C and FIG. 11). The lack of coordinated FAK/Src activation upon ANGPTL4 overexpression may explain the effects observed on motility and migration in these cells.

1. A method for qualifying bladder cancer status in a subject comprising measuring ANGPTL4 in a biological sample from the subject.

2. The method of claim 1, wherein nuclear ANGPTL4 is measured.

3. The method of claim 1, wherein secreted ANGPTL4 is measured.

4. The method of claim 1, wherein full length ANGPTL4 is measured.

5. The method of claim 1, wherein carboxy terminal ANGPTL4 is measured.

6. The method of claim 1, wherein the biological sample is a bladder tissue sample.

7. The method of claim 1, wherein ANGPTL4 is measured by immunoassay.

8. The method of claim 1, wherein ANGPTL4 is measured by immunohistochemistry.

9. The method of claim 1, wherein an elevated level of ANGPTL4 indicates a favorable prognostic bladder cancer status.

10. The method of claim 1, further comprising managing subject treatment based on the status.

11. An ex vivo immobilized cancer cell comprising an ANGPTL4 detection reagent.

12. The ex vivo immobilized cancer cell of claim 11, wherein the cancer cell is a bladder cancer cell.

13. The ex vivo immobilized cancer cell of claim 11, wherein the cancer is immobilized on a glass slide.

14. The ex vivo immobilized cancer cell of claim 11, wherein the ANGPTL4 detection reagent is an antibody that specifically binds ANGPTL4 or a fragment thereof.
